(19) United States
(12) Patent Application Publication
(10) Pub. No.: US 2004/0260200 A1
(43) Pub. Date: Dec. 23, 2004

(54) Medical devices and related methods

(75) Inventor: Frank A. Morello, Pearland, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/821,108

(22) Filed: Apr. 8, 2004

Related U.S. Application Data
(60) Provisional application No. 60/461,495, filed on Apr. 9, 2003.

(51) Int. Cl. A61B 10/00

(52) U.S. Cl. 600/566

(57) ABSTRACT

Medical devices for insertion into a body cavity, such as the thoracic cavity. One version of the medical devices includes an outer needle with side openings in the shaft that communicate with a central passageway of the outer needle, and a stylet having a tapered distal end and a shaft that is configured to be slidably positioned within the passageway of the outer needle. The stylet shaft in this version has different cross-sectional areas at different locations along the stylet shaft. Methods of performing a medical procedure on a subject using the disclosed medical devices. One such procedure is a lung biopsy on a subject.

### CROSS-REFERENCE(S) TO RELATED APPLICATION(S)

This application claims priority to U.S. Provisional Patent Application Ser. No. 60/461,495, filed Apr. 9, 2003, the entire contents of which are expressly incorporated by reference.

### BACKGROUND OF THE INVENTION

1. Field of the Invention

The present invention relates to medical devices for insertion into an anatomical structure, such as a lung. The present invention also relates to methods of obtaining a biopsy using an embodiment of the disclosed medical devices, and to kits that include embodiments of the present medical devices.

2. Description of Related Art

In mammals, both lungs are protected by the ribs in the thoracic cavity. Each lung has a thin, moist lining called the visceral pleura. A tougher lining called the parietal pleura surrounds the inside of the chest wall. Between these two linings is a space that is normally under negative pressure. During an effort to take a breath, the lungs inflate because the chest muscles cause the chest to expand. This causes the negative pressure inside the chest to increase, and actually helps pull the lungs open to become more expanded, taking in more air. When a subject undergoes a needle biopsy of the lung, the needle entering into the lung crosses both pleural linings. Insertion of the needle can disrupt the negative pressure space.

The exact reason for disruption of the negative pressure in the thoracic cavity following needle biopsy of the lung is unknown. Some believe that the needle causes air under positive pressure to enter the thoracic cavity through, or around, the needle. Others think that placing a needle in the lung tears very tiny airways within the lung tissue, where air under positive pressure taken in during a breath can leak out from the lung into the negative pressure space. In any event, if a sufficient quantity of air under positive pressure enters the pleural space, the lung can collapse, resulting in a pneumothorax.

Pneumothorax occurs on the average in 22-45% of people who undergo lung biopsy. Some patients are able to tolerate the pneumothorax, provided the leak of air does not continue, and the air that has leaked in causing loss of negative pressure reabsorbs over time, resulting in restoration of negative pressure. Other patients cannot tolerate a pneumothorax because of lung disease resulting in stiffness of the lung tissue. In addition, advanced age may not let the patient exert the force needed to keep the lung from collapsing, the leak may not seal off quickly enough, and/or the lung may have completely collapsed because of a substantial initial air leakage.

About 5-15% of those who undergo a lung biopsy will need a small plastic tube inserted into their chest to quickly remove the positive air pressure. If this tube is attached to a negative pressure valve, most can have the chest tube removed the next day, when the negative pressure in their chest is restored resulting in resolution of the pneumothorax.

Despite best efforts, the incidence of biopsy-related pneumothorax has not been reduced. The reasons are complicated and poorly understood.

### SUMMARY OF THE INVENTION

The present medical devices, kits and methods are useful for minimizing the incidence of pneumothorax associated with lung biopsy. There are many other uses for the present medical devices, kits and methods, however, besides lung biopsies.

One embodiment of the present medical devices includes an outer needle having a shaft, a passageway, an open end communicating with the passageway, and side openings in the shaft that communicate with the passageway. This embodiment also includes a stylet having a tapered distal end and a stylet shaft configured to be slidably positioned within the passageway of the outer needle. The stylet shaft has different cross-sectional areas at different locations along the stylet shaft.

Another embodiment of the present medical devices includes an outer needle having a shaft with a 16-gauge to 19-gauge outer diameter, a passageway, an open end communicating with the passageway, side openings in the shaft that communicate with the passageway, and a stylet having a distal end and a portion configured to be slidably positioned within the passageway.

A further embodiment of the present medical devices includes an outer needle having a shaft, a passageway, an open end communicating with the passageway, and side openings in the shaft that communicate with the passageway. In this embodiment, two of the side openings are spaced greater than 1 centimeter apart. This embodiment also includes a stylet having a portion configured to be slidably positioned within the passageway.

One embodiment of the present kits includes one of the present medical devices.

One embodiment of the present methods is a method of performing a medical procedure on a subject. The method includes inserting one of the present medical devices into the subject.

Other embodiments of the present medical devices, kits and methods are described below.

### BRIEF DESCRIPTION OF THE DRAWINGS

The following drawings demonstrate certain aspects of the present medical devices and methods. They illustrate by way of example and not limitation.

FIG. 1 is a perspective view of one of the present medical devices, showing an outer needle with a shaft, a passageway, and side openings in the shaft that communicate with the passageway; and a stylet configured to be slidably positioned within the passageway of the outer needle.

FIG. 2 is a perspective view of one of the present medical devices, showing an outer needle with a shaft, a passageway, side openings in the shaft that communicate with the passageway; where the outer needle is attached to a valve that includes an opening that allows the stylet to be slidably positioned within the passageway of the outer needle when the valve is attached to the outer needle; and where the stylet is configured to be slidably positioned within the passageway of the outer needle.

FIG. 3 is a cross-sectional view of one of the present medical devices, showing an outer needle with a shaft, a passageway, and side openings in the shaft that communicate with the passageway; and a stylet configured to be slidably positioned within the passageway of the outer needle, the stylet having different cross-sectional areas at different locations along the stylet shaft.

FIG. 4 demonstrates three different examples of outer needles used in different embodiments of the present medical devices, showing variation in the location of the side openings in the shaft of the outer needle that communicate with the passageway.

FIG. 5 demonstrates a front view of a stylet of one embodiment of the present medical devices, demonstrating different cross-sectional areas of the stylet at different locations along the shaft of the stylet.

FIGS. 6-8 demonstrate stages of one manner of using one of the present medical devices, where the medical device is used to conduct a lung biopsy.

### DESCRIPTION OF ILLUSTRATIVE EMBODIMENTS

The terms “comprise” (and any form of comprise, such as “comprises” and “comprising”), “have” (and any form of have, such as “has” and “having”), and “include” (and any form of include, such as “includes” and “including”) are open-ended linking verbs. Thus, a medical device “comprising” an outer needle having a shaft, a passageway, an open end communicating with the passageway, and side openings in the shaft that communicate with the passageway; and a stylet having a tapered distal end and a stylet shaft configured to be slidably positioned within the passageway of the outer needle, the stylet shaft having different cross-sectional areas at different locations along the stylet shaft, is a medical device that possesses the described outer needle and stylet, but is not limited to possessing only the described outer needle and stylet. For example, the medical device could also include a valve. Similarly, the outer needle is not limited to possessing only the described features (e.g., a shaft, a passageway, etc.). The outer needle could also include, for example, a hub.

The terms “a” and “an” mean one or more than one unless this disclosure explicitly requires otherwise. The term “another” means at least a second or more.

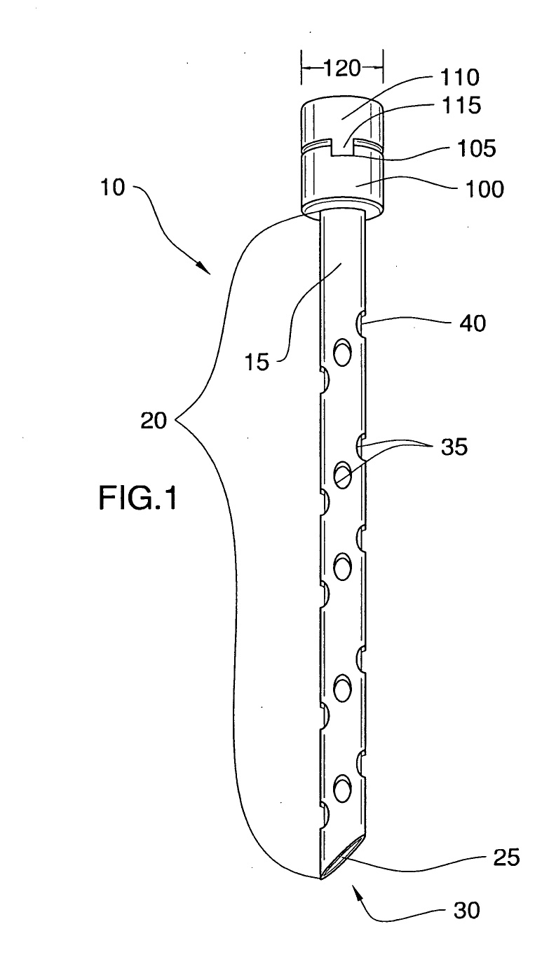

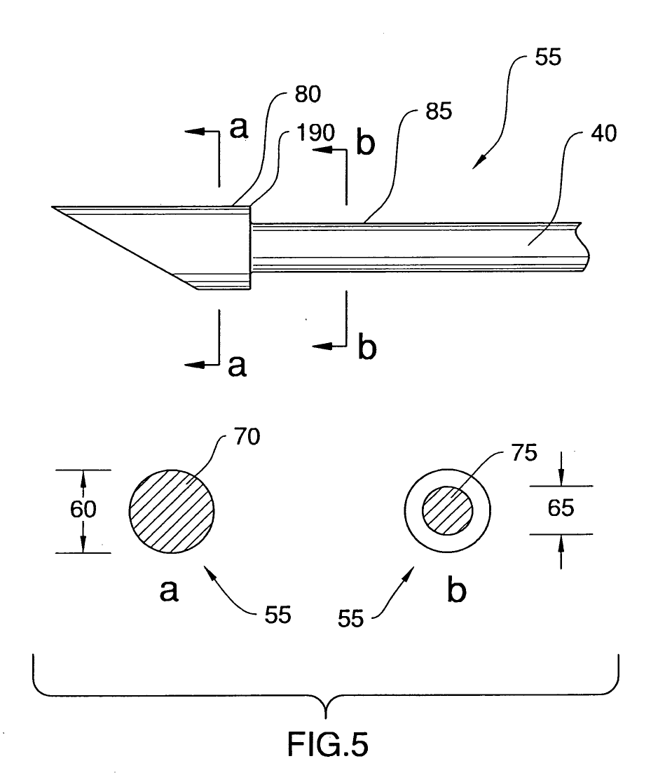

One of the present medical devices is shown in FIG. 1. Medical device 10 includes an outer needle 15 having a shaft 20, a passageway 25, an open end 30 communicating with the passageway, and side openings 35 in the shaft 20 that communicate with the passageway 25. Medical device 10 also includes a stylet 40 having a tapered distal end 90 and a stylet shaft 55 configured to be slidably positioned within the passageway 25 of the outer needle 15. Stylet 40 has different cross-sectional areas (see FIG. 5; 70 and 75) at different locations (see FIG. 5; 80 and 85) along stylet shaft 20.

In the embodiment of medical device 10 shown in FIG. 1, open end 30 of outer needle 15 has a tapered tip. “Open end” means an end that has an opening; the opening need not be centered. The tapered tip may or may not be a sharp tip. In other embodiments of the present medical device, the outer needle may not have a tapered tip.

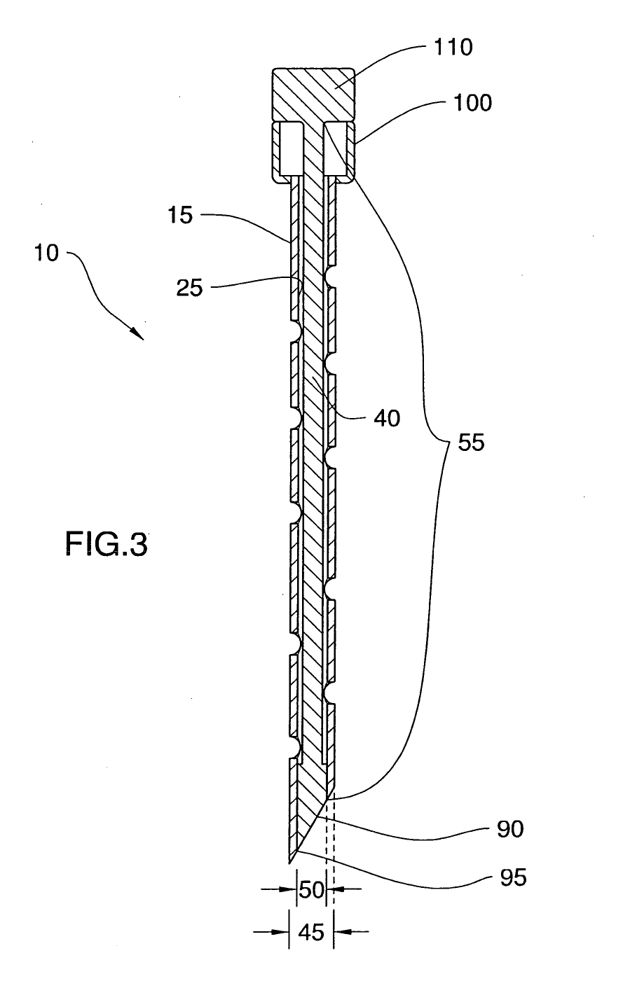

In the embodiment of medical device 10 shown in FIG. 1, distal end 90 (see FIG. 3) of stylet 40 has a sharp tip 95 that fits closely against a portion of the inner surface of passageway 25 of outer needle 15. A tip that fits “closely” against a surface is a tip that will slide easily through the passageway 25 of outer needle 15 but not allow air, fluid, or tissue to interpose between the tip and inner surface of the shaft of the outer needle. In some embodiments, the close fit may be a result of a sharp tip 95 configuration that matches the configuration of the portion of the inner surface of passageway 25 against which it is positioned, resulting in an air-tight fit. The length of the portion of stylet 40 that fits closely against a portion of the inner surface of passageway 25 can be of any length. In one embodiment, that length may be 5 millimeters. In other embodiments, the length may be greater or less than 5 millimeters.

In other embodiments of the present medical device, the stylet may not have a sharp tip that fits closely against a portion of the inner surface of the passageway of the outer needle. For example, in certain embodiments, although stylet 40 may have a distal end 90, the distal end may not include a sharp tip.

As noted above, in some embodiments of the present medical device, the distal end of the stylet 40 includes a sharp tip that fits closely against a portion of the inner surface of the passageway 25 of the outer needle 15. The close fit may or may not be an air-tight fit. In some embodiments, the close-fit is an air-tight fit. The length of stylet 40 that fits closely with the inner surface of the passageway 25 can be of any length. In one embodiment, the length of stylet 40 that fits closely with a portion of the inner surface of the passageway 25 is 5 millimeters. In other embodiments, the length may be greater or less than 5 millimeters.

In the embodiment of medical device 10 shown in FIG. 1, outer needle 15 includes a hub 100. In this context, “includes” means the hub can be permanently attached to the outer needle (and, more specifically, to shaft 20 of outer needle 15), temporarily attached to the outer needle, or integrally formed with the outer needle. Methods of permanent attachment include soldering, welding, and gluing. Methods of temporary attachment include the use of one or more threads, the use of one or more snaps, or the use of other interlocking configurations well known to those of skill in the art. Methods of integral formation (e.g., the hub and outer needle are both part of the same structure) include casting and molding (such as injection molding). Outer needle 15 may be made of any suitable metal or alloy known to those of skill in the art. Hub 100 may be made of the same material. Alternatively, hub 100 may be made of a polymer, such as a medical grade plastic, well known to those of skill in the art.

The configuration of hub 100 can vary with the application and is not limited to the generically-represented hub shown in the figures (the hubs and valves that may be used with the various embodiments of the present medical devices are all represented generically in the figures). Hub styles are well known in the art. For example, hub 100 may include a notch 105 or a similar feature that facilitates attachment of hub 100 to another adjacent hub, such as hub 110 of stylet 40. In other embodiments, outer needle 15 does not include a hub.

In the embodiment of medical device 10 shown in FIG. 1, shaft 55 of stylet 40 includes a hub 110. In this context, “includes” means the hub can be permanently attached to the stylet (and, more specifically, to shaft 55 of stylet 40), temporarily attached to the stylet, or integrally formed with the stylet, in the manners described above. Hub 110 can be composed of any material known to those of skill in the art. For example, the hub can be composed of the same material as the stylet. Alternatively, the two can be made from different materials. For example, stylet 40 can be made of a medical grade metal or alloy, and hub 110 can be made of a medical grade polymer. Hub 110 (as well as hub 100) can be a disposable hub.

Hub 110 may include a male portion 115 configured to mate with notch 105 of hub 100 of outer needle 15. In other embodiments of the present medical devices, other features known to those of skill in the art may be used to facilitate the securing of hub 100 of outer needle 15 with hub 110 of stylet 40.

The dimensions of the present medical devices may be chosen based on the application for which the device will be used. One embodiment of the present medical devices may include an outer needle 15 having a shaft 20 that may be 5 to 15 cm in length. In other embodiments, the length may be greater than 20 cm or less than 5 cm. In some embodiments, shaft 20 may have a length of 10 centimeters (cm). The inner and outer diameters of the outer needle of certain of the present medical devices may also be chosen based on the application for which the device will be used. In some embodiments of the present medical devices, outer diameter 45 of outer needle 15 may range from 1.067 millimeters (mm) (19 gauge; 0.0420 inches) to 1.651 mm (16 gauge; 0.0650 inches), including 17 gauge (1.473 mm; 0.0580 inches) and 18 gauge (1.270 mm; 0.0500 inches) needles. In other embodiments of the present medical devices, outer diameter 45 of outer needle 15 may be less than 1.067 millimeters or greater than 1.651 millimeters (e.g., 15 gauge (1.829 mm; 0.0720 inches) or 14 gauge (2.108 mm; 0.0830 inches)). Chiba needles by Cook, Inc. are suitable for use as the present outer needles.

In embodiments of the present medical devices where outer diameter 45 of outer needle 15 ranges from 1.067 mm (19 gauge; 0.0420 inches) to 1.651 mm (16 gauge; 0.0650 inches), stylet 40 may or may not have different cross-sectional areas at different locations along stylet shaft 55.

In some embodiments of the present medical devices, inner diameter 50 of outer needle 15 may range from 0.686 mm (19 gauge; 0.027 inches) to 1.194 mm (16 gauge; 0.0470 inches). In other embodiments of the present medical devices, inner diameter 50 of outer needle 15 may be less than 0.686 mm or greater than 1.194 mm. In one embodiment of the present medical devices, outer diameter 45 of outer needle 15 may be 1.067 mm (19 gauge; 0.0420 inches), and inner diameter 50 of the outer needle 15 may be 0.686 mm (19 gauge; 0.027 inches).

If the embodiments of the present medical devices include a hub, the diameter of the hub is chosen based on the application for which the device will be used. In some embodiments, such as the embodiment of the medical device 10 shown in FIG. 1, the hubs of outer needle 15 and stylet 40 may each have a diameter 120 that is larger than the diameters of both outer needle 15 and stylet 40.

Similarly, the dimensions of the stylet of certain of the present medical devices may be chosen based on the application for which the device will be used. In certain embodiments, shaft 55 of stylet 40 may be 10, 15, or 20 cm in length. In other embodiments, the length of the shaft 55 of stylet 40 plus the length of the tip of the shaft of stylet 40 is equal to the length of shaft 20 of outer needle 15 plus the length of hub 100 of the outer needle 15 plus the length of valve 140.

Shaft 55 of stylet 40 may or may not have a passageway. In one embodiment, shaft 55 is solid and does not include a passageway. The differing diameters (60, 65) of shaft 55 of stylet 40 may also be chosen based on the application for which the device will be used. As discussed above, stylet shaft 55 has different cross-sectional areas (70, 75) at different locations (80, 85) along the stylet shaft 55. For example, stylet shaft 55 may have an outer diameter that ranges from 0.686 mm (0.027 inches) to 1.194 mm (0.0470 inches), and a cross-sectional area that ranges from 0.370 mm² (0.0006 inches²) to 1.120 mm² (0.0017 inches²).

Side openings 35 may be provided in shaft 20 of outer needle 15 using any suitable method. For example, side openings 35 may be drilled, or they may be cut using, for example, a laser or chemical etching. Suitable materials for use as outer needle 15 include stainless steel, tungsten, or any other suitable biocompatible material known to those skilled in the art. The present stylets may be made of any of the same materials.

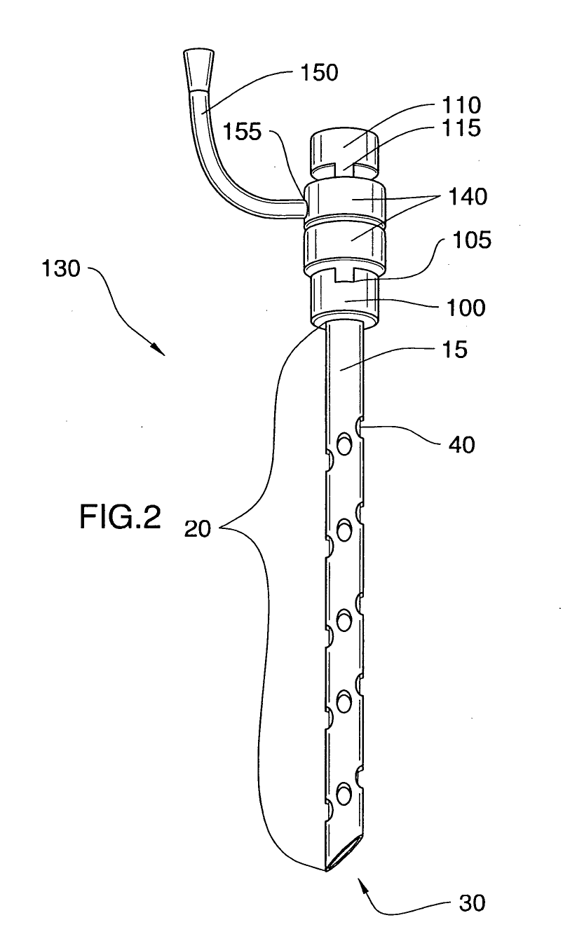

FIG. 2 demonstrates a side view of another of the present medical devices, where outer needle 15 is configured to attach to a valve 140 that includes an opening (not visible) that allows stylet 40 to be slidably inserted into the passageway (see FIG. 3; 25) of outer needle 15. Hub 110 of stylet 40 can contact valve 140 at the point of full insertion of stylet 40 into the passageway of outer needle 15. Valve 140 may be any suitable valve known to those of skill in the art, including those capable of producing an airtight seal. Outer needle 15 and stylet 40 may be configured to be attached to valve 140 in any fashion known to those of skill in the art. For example, outer needle 15 may be configured to attach to hub 110, where hub 110 is configured to interlock with valve 140 in any manner known to those of skill in the art, including by luer locking. Valve 140 may include a side port 155 and attached tubing 150 that is configured to attach to an external source of suction, such as wall suction. The external source of suction may, for example, be applied during a biopsy using one of the present medical devices such that negative pressure is maintained within passageway 25 of outer needle 15 during the biopsy procedure.

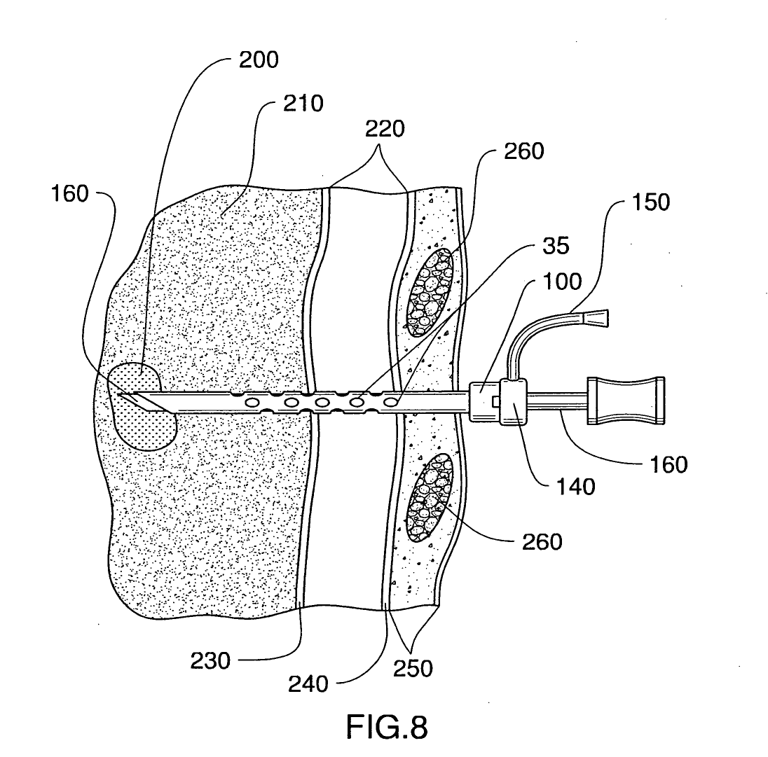

The outer needle of certain of the present medical devices may be configured to allow a biopsy needle to be slidably inserted into its passageway. The biopsy needle can, for example, be a lung biopsy needle (see FIG. 8; 160). Such an outer needle may be part of an embodiment of the present medical devices that is configured to attach to a valve 140 that includes a sideport 155 and attached tubing 150 that can be hooked up to a source of negative pressure such that negative pressure will be maintained in the passageway of outer needle 25 during the lung biopsy. As those of skill in the art will understand, the size of the biopsy needles suited for use with the present medical devices may be based on the size of the outer needle with which they will be used. For example, a biopsy needle configured for use with an 18-gauge outer needle may be, for example, a 20-gauge or 22-gauge needle. In this regard, a 22-gauge or a 20-gauge biopsy needle may be useful for obtaining an aspirate tissue sample. In this same regard, a 20-gauge biopsy needle may also be useful for obtaining a core tissue sample.

FIG. 3 demonstrates a cross-sectional view of medical device 10 shown in FIG. 1. Stylet 40 is configured to be slidably positioned within passageway 25 of outer needle 15. Stylet shaft 55 of medical device 10 shown in FIG. 3 has different cross-sectional areas (see 70 and 75 in FIG. 5) at different locations along stylet shaft 55. In the embodiment of medical device 10 shown in FIG. 3, distal end 90 of stylet 40 includes a sharp tip that is configured to fit closely against a portion of the inner surface of passageway 25.

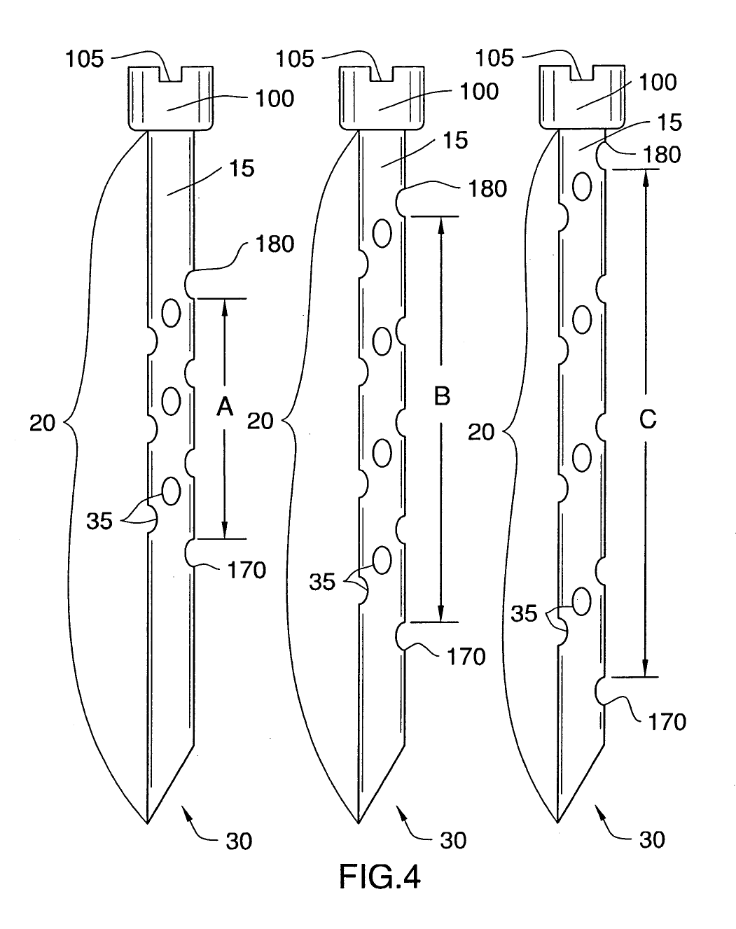

FIG. 4 demonstrates three different examples of outer needles used in different embodiments of the present medical devices, showing variation in the location of side openings 35 in shaft 20 of outer needle 15 that communicate with passageway 25. More specifically, in these three examples, side openings 35 are spaced along different lengths of shaft 20 of outer needle 15. For example, distance A between the inner edge of the side openings that are spaced farthest from each other in the first example is shorter than distances B and C in the second and third examples. The spacing of side openings 35 may be chosen based on the application for which the device will be used. Distances A, B, and C between the inner edges of the side openings that are spaced farthest from each other in the three examples shown in FIG. 4 may be at least 2 cm, at least 5 cm, and at least 7 cm, respectively, provided those distances are suited to the application to be performed. As another example, in some embodiments of the present medical devices, two side openings in shaft 20 of outer needle 15 are spaced greater than 1 cm apart. This means that the distance between the inner edges of the two side openings (as shown, for example, in FIG. 4) is greater than 1 cm. In other embodiments of the present medical devices, the side openings are spaced 0.5 cm apart. In embodiments where two side openings are spaced greater than 0.5 cm or 1 cm apart, the stylet may or may not have different cross-sectional areas at different locations along the shaft of the stylet, and the outer diameter of the outer needle ranges from 16-gauge to 19-gauge.

Side openings 35 can be positioned in shaft 20 of outer needle 15 in any suitable location. Depending on the application, it may be desirable to locate more side openings along a particular portion of the shaft. For example, if a lung biopsy is to be performed, it will be desirable to locate a higher concentration of side openings in the section of the shaft that will be positioned within the pleural cavity so as to ensure that negative pressure in that cavity can be best maintained during the procedure.

The location of side opening 170, which is the side opening nearest to open end 30 of outer needle 15, may be chosen based on the application for which the medical device will be used. In one embodiment, the outer edge of side opening 170 may be 0.5 cm from open end 30 of outer needle 15. Likewise, the location of side opening 180, which is the side opening located nearest to hub 100 of outer needle 15, may be chosen based on the application for which the device will be used. In one embodiment, the outer edge of side opening 180 may be 2.0 cm from the bottom (or distal end) of hub 100.

FIG. 5 depicts one embodiment of stylet 40 of medical device 10, and shows that the stylet may have different cross-sectional areas 70 and 75 at different locations 80 and 85 along stylet shaft 55. In the embodiment of medical device 10 shown in FIG. 5, shaft 55 of stylet 40 changes angle at position 190, resulting in shaft 55 having two different cross-sectional areas 70 and 75 at different locations 80 and 85. That change of angle may be sharp, as shown in FIG. 5, or it may be rounded (not shown) or tapered (not shown). For example, in other embodiments of the medical device, shaft 55 of stylet 40 may have a sloping appearance along the portion of the shaft where the cross-sectional area of the shaft 55 varies. Furthermore, there may be multiple positions along the stylet 40 (and, more specifically, along shaft 55) where the cross-sectional area of the stylet (and, more specifically, the stylet shaft) varies. Cross-sectional area 70 is taken along line a-a in FIG. 5, and cross-sectional area 75 is taken along line b-b in FIG. 5.

The cross-sectional area of the present stylets can be round, as shown in FIG. 5, or any other configuration that works with the configuration of the inner surface of the passageway of the outer needle (e.g., oval). The cross-sectional configuration of the stylet can also vary along the length of the shaft of the stylet. One of skill in the art would be familiar with different configurations suitable for cross-sections of the stylet and the outer needle.

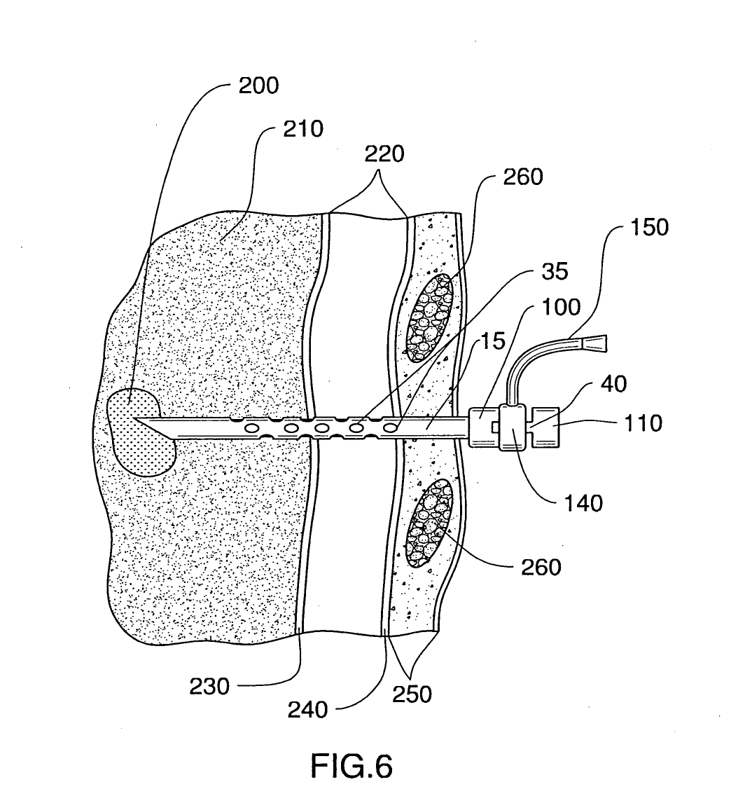

The present medical devices may be used to conduct a medical procedure on a subject. For example, FIGS. 6-8 demonstrate stages of one manner of using one of the present medical devices during a procedure for conducting a lung biopsy of a subject. The medical device that is used is medical device 130 shown in FIG. 2.

FIG. 6 demonstrates the position of medical device 130 within a subject having a lung lesion 200 in lung 210, during an initial phase of the biopsy. Medical device 130 includes an outer needle 15 with side openings 35 in its shaft. Outer needle 15 includes hub 100. Medical device 130 also includes valve 140, which has a side tubing 150 configured to attach to an external source of negative pressure; and stylet 40 having hub 110. Stylet 40 is configured to be slidably inserted into the passageway of outer needle 15. In FIG. 6, medical device 130 has been inserted between ribs 260 through chest wall 250, through pleural lining 210 of the chest wall, across pleural space 220, through pleural lining 230 of the lung, and into tumor 200 located within lung 210. Computed tomographic, ultrasonic, or fluoroscopic guidance may be used to direct placement of the medical device into the lesion. During insertion of medical device 130, sideport 150 of valve 140 may be connected to a source of negative pressure in an effort to prevent a pneumothorax. The stylet may be maintained in position within the passageway of outer needle 15 during insertion of medical device 130 into the lung lesion.

The present medical devices may be advantageously used in, for example, procedures where isolated suction is needed as the procedure is taking place—e.g., as the medical device is being positioned. One advantage results from configuring the stylet used to have (a) a portion that fits closely against a portion of the inner surface of the outer needle that is used, and (b) a portion that does not fit as closely against the inner surface of the outer needle (see, e.g., FIG. 3). By also providing side openings in the shaft of the outer needle, air outside of the side openings can then be suctioned (a) into the space between the outside of the stylet shaft and the surface of the passageway of the outer needle and (b) out of the valve or other apparatus that is facilitating the suctioning (c) without drawing air in through the open end of the medical device. The side openings may be strategically positioned depending on the anatomy of the subject and the nature of the procedure to precisely effect where the suction will occur.

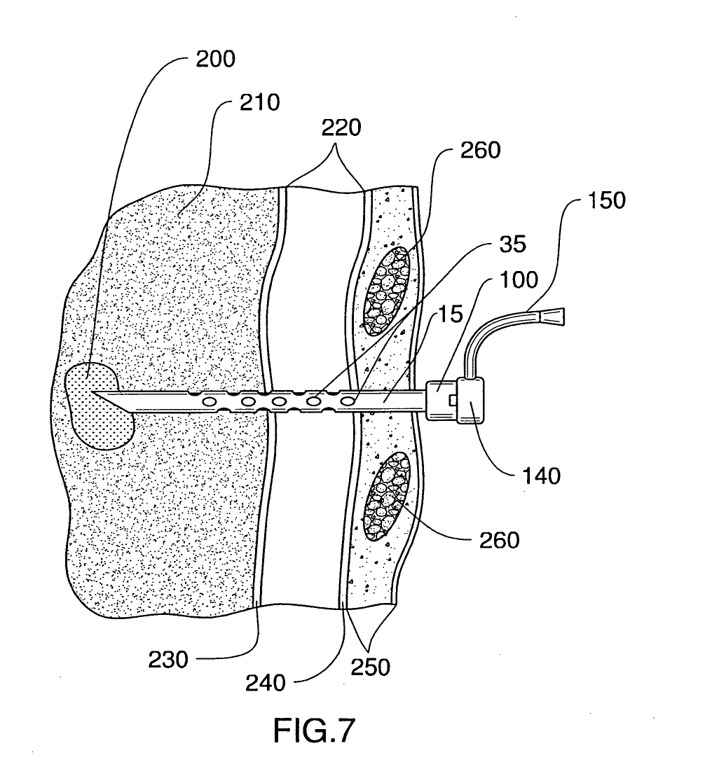

Following insertion of medical device 130 into lung lesion 200, stylet 40 is removed (FIG. 7). Negative pressure may be maintained through sideport 150 of valve 140 in an effort to minimize the risk of development of pneumothorax. Following removal of stylet 40, a lung biopsy needle 160 is inserted through valve 140 and outer needle 15 and into lung lesion 200. Computed tomographic, ultrasonic, or fluoroscopic guidance may be used to assist in positioning the tip of lung biopsy needle 160 into lung lesion 200. Negative pressure may be maintained through sideport 150 of valve 140 during the biopsy of lung lesion 200 in an effort to minimize the risk of development of pneumothorax. Following the biopsy of lung lesion 200 with lung biopsy needle 160, which biopsy may produce an aspirate sample or a core sample (and a core sample may be taken after an aspirate sample is taken and the lesion's location confirmed), the lung biopsy needle may be removed and stylet 40 repositioned within the passageway of outer needle 15. During removal of medical device 130, negative pressure may be maintained through sideport 150 of valve 140 to minimize risk of pneumothorax. One of skill in the art would be familiar with variations of this embodiment of using the depicted medical device that fall within the scope of the claims below and their equivalents. The present medical devices can also be used to conduct other medical procedures, such as biopsies of organs other than the lung.

Kits containing the present medical devices may be sold. One embodiment of such a kit may include an enclosure (such as a plastic bag that is sealed) and one of the present medical devices. Thus, the kit may include one of the present outer needles (with or without a hub, and with or without a valve attached to the hub) and one of the present stylets. In other embodiments, one or more of the present biopsy needles sized to fit within the passageway of the subject outer needle may be included. Other embodiments of such kits may also include instructions for use of the medical device.

The following example is included to demonstrate one embodiment of the present medical devices.

### EXAMPLE

Lung biopsies on 10 large dogs (mean size 30 kilograms) were performed using one embodiment of the present medical devices. The outer needle that was used a standard 5-cm long, 18-gauge Chiba needle (Cook, Inc.). Several side openings were cut into the outer needle that communicated with the outer needle's passageway. The outer needle included a hub to which an airtight valve was luer-locked, and the sideport of the valve was attached to a 100 mm-Hg of wall suction. A stylet for a 10-cm-long, 19-gauge Chiba needle (Cook, Inc.) was cut to fit the length of the outer needle plus the length of the attached valve. Air entering the outer needle could travel through its passageway, around the stylet, and out the sideport of the valve to the wall suction container. The valve was configured such that it could open around biopsies needles that were placed through it and the outer needle, and then close around them to maintain the suction during tissue sampling. In this regard, aspirate samples were taken using a coaxial 22-gauge needle, and core tissue samples were taken with a coaxial 20-gauge core needle with interval CT scans taken to document pneumothorax. Four pneumothoraces occurred.

The present medical devices can be made and used without undue experimentation in light of the disclosure. The medical devices described above need not be made in the exact disclosed forms, or combined in the exact disclosed configurations to fall within the scope of the claims and their equivalents. Instead, it is possible to make substitutions, modifications, additions and/or rearrangements of the features disclosed above without deviating from the scope of the claims and their equivalents. Further, although the present methods can be practiced using the specific techniques disclosed above, such methods can also be practiced using other techniques.

The appended claims are not to be interpreted as including means-plus-function limitations, unless such a limitation is explicitly recited in a given claim using the phrase(s) “means for” and/or “step for,” respectively.

I claim:

1. A medical device comprising:
an outer needle having a shaft, a passageway, an open end communicating with the passageway, and side openings in the shaft that communicate with the passageway; and
a stylet having a tapered distal end and a stylet shaft configured to be slidably positioned within the passageway of the outer needle, the stylet shaft having different cross-sectional areas at different locations along the stylet shaft.

2. The medical device of claim 1, where the distal end of the stylet comprises a sharp tip that fits closely against a portion of the inner surface of the passageway.

3. The medical device of claim 1, where two side openings in the shaft of the outer needle are spaced greater than 1 centimeter apart.

4. The medical device of claim 1, where the side openings located farthest from each other are at least 2 centimeters apart.

5. The medical device of claim 1, where the side openings located farthest from each other are at least 5 centimeters apart.

6. The medical device of claim 1, where the side openings located farthest from each other are at least 7 centimeters apart.

7. The medical device of claim 1, where the outer needle includes a hub.

8. The medical device of claim 7, where the hub includes a notch.

9. The medical device of claim 8, where the stylet includes a hub.

10. The medical device of claim 9, where the hub of the stylet includes a male portion configured to mate with the notch in the hub of the outer needle.

11. The medical device of claim 1, where the outer needle is configured to attach to a valve that includes an opening that allows the stylet to by slidably positioned within the passageway of the outer needle when the valve is attached to the outer needle.

12. The medical device of claim 1, where the outer needle is configured to allow a lung biopsy needle to be slidably inserted into the passageway.

13. A medical device comprising:
an outer needle having a shaft with a 16-gauge to 19-gauge outer diameter, a passageway, an open end communicating with the passageway, and side openings in the shaft that communicate with the passageway; and
a stylet having a distal end and a portion configured to be slidably positioned within the passageway.

14. The medical device of claim 13, where the distal end of the stylet comprises a sharp tip that fits closely against a portion of the inner surface of the passageway.

15. The medical device of claim 13, where two side openings in the shaft of the outer needle are spaced greater than 1 centimeter apart.

16. The medical device of claim 13, where the side openings located farthest from each other are at least 2 centimeters apart.

17. The medical device of claim 13, where the side openings located farthest from each other are at least 5 centimeters apart.

18. The medical device of claim 13, where the side openings located farthest from each other are at least 7 centimeters apart.

19. The medical device of claim 13, where the outer needle includes a hub.

20. The medical device of claim 19, where the hub includes a notch.

21. The medical device of claim 20, where the stylet includes a hub.

22. The medical device of claim 21, where the hub of the stylet includes a male portion configured to mate with the notch in the hub of the outer needle.

23. The medical device of claim 13, where the outer needle is configured to attach to a valve that includes an opening that allows the stylet to be slidably positioned within the passageway of the outer needle when the valve is attached to the outer needle.

24. The medical device of claim 13, where the outer needle is configured to allow a lung biopsy needle to be slidably inserted into the passageway.

25. A medical device comprising:
an outer needle having a shaft, a passageway, an open end communicating with the passageway, and side openings in the shaft that communicate with the passageway, two of the side openings being spaced greater than 1 centimeter apart; and
a stylet having a portion configured to be slidably positioned within the passageway.

26. The medical device of claim 25, where the side openings located farthest from each other are at least 2 centimeters apart.

27. The medical device of claim 25, where the side openings located farthest from each other are at least 5 centimeters apart.

28. The medical device of claim 25, where the side openings located farthest from each other are at least 7 centimeters apart.

29. The medical device of claim 25, where the outer needle includes a hub.

30. The medical device of claim 29, where the hub includes a notch.

31. The medical device of claim 30, where the stylet includes a hub.

32. The medical device of claim 31, where the hub of the stylet includes a male portion configured to mate with the notch in the hub of the outer needle.

33. The medical device of claim 25, where the outer needle is configured to attach to a valve that includes an opening that allows the stylet to be slidably positioned within the passageway of the outer needle when the valve is attached to the outer needle.

34. The medical device of claim 25, where the outer needle is configured to allow a lung biopsy needle to be slidably inserted into the passageway.

35. A method of performing a medical procedure on a subject, the method comprising inserting the medical device of any of claims 1, 13 and 25 into the subject.

36. The method of claim 35, further defined as a method of obtaining a lung biopsy in a subject.

37. The method of claim 35, where the medical device is inserted into a lung of the subject.

38. The method of claim 35, where inserting the medical device further comprises connecting the medical device to an external source of suction.

39. The method of claim 35, further defined as a method of preventing a pneumothorax in a subject.
